(19) United States
(12) Patent Application Publication
(10) Pub. No.: US 2018/0059124 A1
(43) Pub. Date: Mar. 1, 2018

(54) APPARATUS AND METHOD FOR EARLY IDENTIFICATION OF tPA RESISTANCE FOR CLOT DISSOLUTION IN PATIENTS

(71) Applicant: Mubin I. Syed, Springfield, OH (US)

(72) Inventor: Mubin I. Syed, Springfield, OH (US)

(73) Assignee: Mubin I. Syed, Springfield, OH (US)

(21) Appl. No.: 15/246,155

(22) Filed: Aug. 24, 2016

(51) Int. Cl. G01N 33/86; G01N 33/96; G01N 33/49

(52) U.S. Cl. CPC G01N 33/86; G01N 33/96; G01N 2333/9726; G01N 2800/52; G01N 33/4905

(57) ABSTRACT

Recombinant tissue plasminogen activator (tPA) is a lytic medication in wide use to treat thrombotic disorders such as stroke, myocardial infarction (MI), and pulmonary embolism (PE). It is also used “off-label” for the treatment of deep venous thrombosis (DVT) and acute peripheral arterial thrombosis/embolism. It has been shown in multiple studies that in approximately 25% of the individuals receiving the treatment the drug is ineffective. Start of the tPA treatment for these patients results in delay of start of alternate treatment for these patients which may be life saving for them. In the past there has been no test to find the effectiveness of the treatment and identify the individuals for whom the treatment will be ineffective. What is disclosed is a method of testing for effectiveness of the tPA treatment, that is testing for thrombolytic resistance, using a sample of the individual's blood via coagulation analysis.

### BACKGROUND

### 1. Field

The invention relates to identifying drug effectiveness in treatment of patients with blood clots using a recombinant tissue plasminogen activator (tPA).

### 2. Related Art

FIG. 1 shows the structure of a blood clot 100. Platelets in the blood 101 get “turned on” by triggers released when any blood vessel is damaged. They stick to the walls in the area and each other, changing shape to form a plug that fills in the broken part to stop blood from leaking out of the vessel. Proteins in the blood called clotting factors cause a rapid chain reaction, causing a dissolved substance in the blood to turn to long strands of fibrin 102. These get tangled up with the platelets in the plug to create a net that enhances and sustains the blood clot 100.

A stroke occurs when a vessel in the brain ruptures or is blocked by a blood clot. Stroke medical treatments work to either open the blockage or treat the rupture. Medical advances have greatly improved survival rates from stroke treatments during the last decade. But the chances of survival are even better if the stroke is identified and treated immediately.

There are two types of strokes: hemorrhagic or ischemic. An ischemic stroke occurs as a result of an obstruction within a blood vessel supplying blood to the brain. It accounts for 87 percent of all stroke cases. A hemorrhagic stroke occurs when a weakened blood vessel ruptures and spills blood into brain tissue.

The only FDA approved drug for treatment of ischemic strokes is tissue plasminogen activator (tPA, also known as IV rtPA, given through an IV in the arm). tPA works by dissolving the clot and improving blood flow to the part of the brain being deprived of blood flow. If administered within 3 hours (and up to 4.5 hours in certain eligible patients), tPA may improve the chances of recovering from a stroke. A significant number of stroke victims don't get to the hospital in time for tPA treatment; this is why it's so important to identify a stroke immediately.

Thrombolytic medications are also approved for the immediate treatment of heart attack. Here again the most commonly used drug for thrombolytic therapy is tissue plasminogen activator (tPA), but there are other drugs that can do the same thing but they have not been found to be as effective as tPA.

There is a better chance of surviving and recovering from certain types of heart attacks if the patient receives a thrombolytic drug within 6-12 hours after the heart attack starts. Ideally, the thrombolytic medications are administered within the first 30 minutes after arriving at the hospital for treatment.

Heart attacks are usually caused when a blood clot blocks the arteries to the heart. This can cause part of the heart muscle to dies due to a lack of oxygen being delivered by the blood.

Thrombolytic work by dissolving a major clot quickly. This helps restart blood flow to the heart and helps prevent damage to the heart muscle. Thrombolytics can stop or reduce the intensity of a heart attack that would otherwise be deadly.

The drug restores some blood flow to the heart in most patients. However, the blood flow may not be completely normal and there may still be a small amount of muscle damage. Additional therapy, such as cardiac catheterization with angioplasty or stenting (percutaneous coronary intervention or PCI), may be needed in most heart attack cases.

Tissue plasminogen activator (tPA) is the approved medication to treat all major thrombotic disorders such as myocardial infarction (MI), ischemic stroke (IS), thrombosis of dialysis grafts and central lines, and pulmonary embolus (PE). Though the treatment has been found to be very effective in most cases where the tPA acts to dissolve or reduce the size of the blood clot, in a section of individuals, between 20 and 30% treated by tPA show resistance to treatment.

Since many patients with MI, IS and submassive to massive PE are initially treated using tPA, the resistance to such treatment in the 20 to 30% of the patient population put them at risk due to delay in identifying the lack of responsiveness. Typically, such delays can be as much as 1 to 2 hours or longer, which can result in permanent damage to the organs of the patient or even cause loss of life.

Alternative treatments for MI not involving tPA includes the use of mechanical devices such as percutaneous coronary intervention (PCI) which includes percutaneous transluminal coronary angioplasty and stenting, as well as percutaneous mechanical thrombectomy or alternatively coronary artery bypass graft surgery (CABG). Alternative treatment for IS not involving tPA includes the use of mechanical thrombectomy/embolectomy devices. Alternative treatments for PE not involving tPA includes mechanical thrombectomy/embolectomy devices, mechanical thromboaspiration and surgical thrombectomy/embolectomy. The same is true for DVT and acute peripheral arterial thrombosis/embolism (in which case catheter-directed tPA infusion would be replaced by either 1) a percutaneous mechanical or aspiration type thrombectomy/embolectomy and 2) possibly percutaneous transluminal angioplasty or stenting versus 3) surgical thrombectomy/embolectomy or bypass for clot in the arterial system. The percutaneous mechanical treatments for MI, IS or submassive to massive PE are much more involved than the treatment using IV tPA but should be considered if tPA treatment is found to be ineffective (by this test) or contraindicated in general.

Accordingly, what is needed, in treatment of thrombotic disorders, is a method and a testing system that can let the medical professionals know in advance if treatment with tPA will or will not be effective for any individual patient requiring treatment with tissue plasminogen activator (tPA).

### SUMMARY

The following summary of the invention is included in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

In accordance with one aspect of the invention, a method is disclosed that includes drawing an amount of a fresh blood sample from a patient sufficient to fill two cuvettes; splitting the drawn blood sample into two blood sample portions and filling the two cuvettes with the two blood sample portions; mixing a small pre-defined amount of the tissue plasminogen activator (tPA) into one of the two cuvettes; placing the two cuvettes into a blood coagulation tester to test clotting characteristics of the two sample blood portions, wherein a set of traces of the clotting function of blood as the blood sample portions in the two cuvettes clot is generated; comparing the set of traces with normal blood that is non-resistant to tPA; and determining a tPA response of the patient based on the comparing.

The blood coagulation tester may be a Sonoclot® Analyzer.

The set of traces may be used to identify the activated clotting time (ACT) and the clot rate of the blood sample portions.

The values of ACT and clot rate may be extracted from the set of traces.

The set of traces may be compared with a Sonoclot® signature trace.

The set of traces may be compared with a Sonoclot® signature trace and a Sonoclot® trace of normal tPA responsive blood with a pre-determined amount of tPA added.

The method may further include preparing the tPA for efficacy testing.

Preparing the tPA for efficacy testing may include reconstituting cathflo activase; and diluting the solution by adding normal saline. Reconstituting cathflo activase may create a 2 mg of tPA/2 mL solution.

Diluting the solution may dilute the solution by 0.25, and adding normal saline may include adding 6 mL of normal saline.

Mixing a small pre-defined amount of the tissue plasminogen activator (tPA) into one of the two cuvettes may include adding 3.3 μL of the 0.25 diluted solution to 326.7 μL of the one of the two blood sample portions in the one of the two cuvettes.

The fresh blood sample may be drawn from the patient when they are experiencing a blood vessel clot. The blood vessel clot may be a heart attack or stroke. The blood vessel clot may be a pulmonary embolism (PE), a myocardial infarction (MI), an arterial thrombosis, or a deep vein thrombosis (DVT). The patient may be experiencing a Pulmonary Embolism (PE), a myocardial infarction (MI), an arterial thrombosis, or a deep vein thrombosis (DVT) related to the blood vessel clot.

The method may further include administering an effective amount of tPA if the tPA response is that the patient is not thrombolytic resistant.

The method may further include performing an alternate interventional procedure if the tPA response is that the patient is thrombolytic resistant.

In accordance with another aspect of the invention, a blood coagulation tester is disclosed that includes a first cuvette for receiving a normal blood sample from a patient; a second cuvette for receiving a mixture of tPA and a blood sample the patient; a probe in each of the first cuvette and the second cuvette; an electomechanical transducer coupled to each of the probes in the first cuvette and the second cuvette to move the probes in the first and second cuvettes; electronic drive and detection circuitry coupled to each of the electromechanical transducers configured to cause each of the electromechanical transducers to move the probes in the first and second cuvettes and to measure resistance to movement of the probes in the first and second cuvettes; and a microcomputer coupled to the electronic drive and detection circuitry to process the measured resistance and output a set of traces indicating the clotting function of the normal blood sample in the first cuvette and the mixture of the tPA and the blood sample in the second cuvette, wherein the set of traces are configured to be compared to determine whether the blood sample is thrombolytic resistant.

The blood coagulation tester may be a Sonoclot® Analyzer. The blood coagulation tester may be a Thrombo Elastography (TEG®) or a Rotational thromboelastrometry (ROTEM®).

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

FIG. 1 is a diagram 100 of a blood clot that shows the components of the clot.

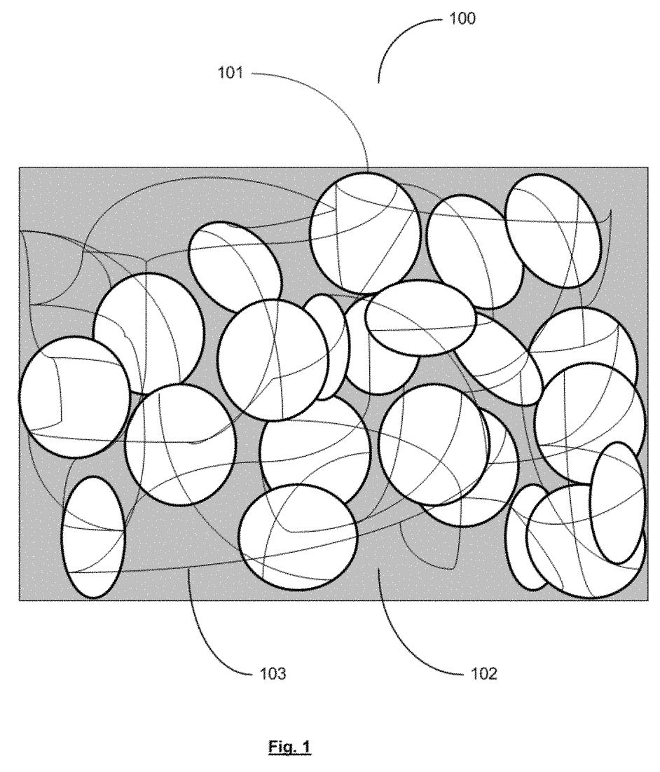

FIG. 2 is a schematic diagram of a Sonoclot® Coagulation & Platelet Function Analyzer in accordance with embodiments of the invention.

FIG. 3 illustrates a typical trace of a Sonoclot® Coagulation & Platelet Function Analyzer of a good blood sample.

FIG. 3A illustrates a plot of a blood sample with hyberfibronolysis using the Sonoclot® Coagulation & Platelet Function Analyzer.

FIG. 4 illustrates exemplary electrical traces from a Sonoclot® Coagulation & Platelet Function Analyzer of typical, not resistant to tPA, blood, with no tPA addition, and with 0.25 tPA addition in accordance with embodiments of the invention.

FIG. 5 illustrates exemplary electrical traces of clotting from a Sonoclot® Coagulation & Platelet Function Analyzer of non-typical, resistant to tPA blood, with no tPA addition, with 0.25 tPA addition and with 0.5 tPA addition in accordance with embodiments of the invention.

FIG. 6 is a flow diagram of a method for identification of tPA resistance for clot dissolution in accordance with embodiments of the invention.

### DETAILED DESCRIPTION

Embodiments of the invention are directed to a new test method developed that uses a Sonoclot® Coagulation & Platelet Function Analyzer or similar equipment for point of care coagulation testing of patient blood (e.g., Thrombo Elastography or TEG® equipment developed by Hemonetics corporation, Brain Tree, Mass., and Rotational thromboelastrometry or ROTEM® equipment developed by TEM international GMBH, Munich, Germany) to test for individual patients resistance to tissue plasminogen activator (tPA) as a drug used to treat conditions caused by arterial blood clots including heart attacks (MI), ischemic strokes, chest pain at rest (unstable angina), blood clots in the lungs (pulmonary arterial thrombosis or embolus (PE)), and other less common conditions involving blood clots. In embodiments using other machines similar to the Sonoclot® machine that provide entire hemostasis process information, as understood by persons of ordinary skill in the art, some modifications to the disclosed testing methods and conditions may be needed to optimize the result using these machines.

In embodiments of the invention, the process is done by withdrawing a small amount of the patient's blood and mixing it with a pre-determined amount of tPA and testing the clot initiation and clotting rate over a short time period of less than 10 minutes using electrical traces from Sonoclot® Coagulation & Platelet Function Analyzer, of typical not resistant to tPA, clotting blood with no tPA addition, and blood clotting with 0.25 tPA added. The clotting characteristics of the patient's without tPA blood is compared to the patient's blood with added tPA, for identifying the effect of the tPA on the clotting c characteristics. This provides an indication of the resistance to the drug for the individual.

Since the time taken for the test is of the order of five to ten minutes and the blood draw volume and drug quantity used are small, embodiments of the invention is able to provide the required knowledge base to the physician on the applicability of using tPA, for the patient being treated, to optimize the treatment methodology and reduce the cost of wasted time and wasted drug use.

Recombinant tissue plasminogen activator (tPA) is a lytic medication in wide use to treat thrombotic disorders such as stroke and myocardial infraction. It has been shown in multiple studies that in approximately 25% of the individuals receiving the treatment the drug is ineffective. Start of the tPA treatment for these patients results in delay of start of alternate treatment for these patients which may be life saving for them. In the past there has been no test to find the effectiveness of the treatment and identify the individuals for whom the treatment will be ineffective. What is disclosed is a method of testing for effectiveness of the tPA treatment, that is testing for thrombolytic resistance, using a sample of the individual's blood via coagulation analysis.

Patients who are experiencing an ischemic stroke (IS), heart attack (MI), PE or any type of blood vessel clot are given a thrombolytic to break up the clots within their blood. In fact, these are among the top causes of death including #1 rank Coronary Artery Disease (CAD) with or without associated myocardial infarction), #3 rank Stroke and Pulmonary Embolism (PE) are treated with tPA. As indicated before the drug administered has varying efficacy in practice depending on the physiology of the patient due to tPA resistance. The tPA resistance affects 22 to 30% of patients treated. It would be a very useful thing if early identification of the tPA resistance, which can prevent permanent damage to and even death of the patient. The early identification is a check to see if a drug will have the desired effect on a patient before starting a course of therapy, so as avoid an ineffective therapy and to direct the patient to an effective alternative therapy. It will also avoid unnecessary expenses due to time and drug wastage. The current invention provides a check of the responsiveness of the patient to the use of iPA as a treatment.

In one embodiment, the method disclosed herein uses a Sonoclot® Coagulation & Platelet Function Analyzer (Sonoclot® Analyzer), which is a versatile instrument for measuring coagulation and platelet function. The Sonoclot® Analyzer provides information on the entire hemostasis process including coagulation, fabrin gel formation, clot retraction (platelet function) and fibrinolysis. The Sonoclot® Analyzer generates a continuous graph (known as the Sonoclot® Signature), and also provides results on the actual clot formation time (ACT), and the rate of fibrin polymerization (Clot RATE) for identifying numerous coagulopathies. (The TEG® and ROTEM® procedures and machines which are alternates used to perform the coagulation analysis function, will each have their own signature traces which will have to be evaluated to identify and evaluate the effect of tPA as has been done for the Sonoclot® process. Since the preferred process for the testing for tPA sensitivity has been done using the Sonoclot® machine that procedure and analysis is what is described in detail below)

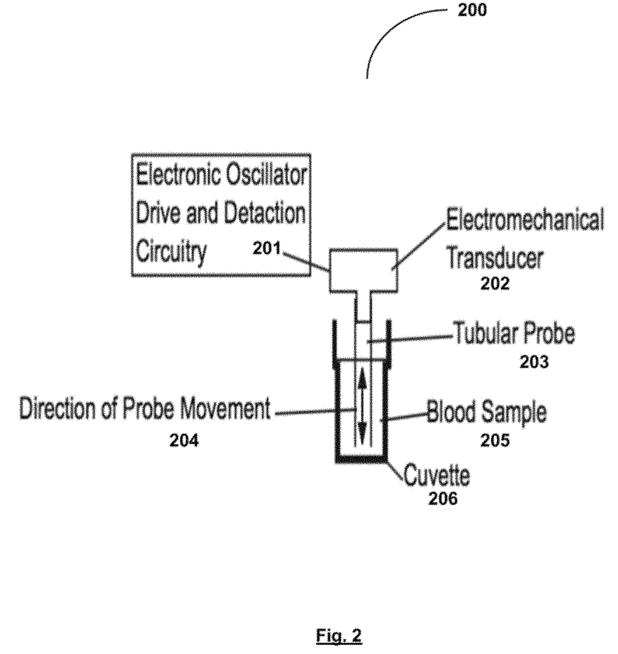

FIG. 2 shows the functional diagram of the working of a Sonoclot® Analyzer 200. The operational mechanism in the Sonoclot® Analyzer 200 is a mechanical hollow probe 203 that is connected to an electrical transducer 202 which drives it up and down within a blood sample 205 inside a cuvette 206 and an electronic drive and detection circuitry 201. Over time the blood sample 205 in the cuvette 206 goes through the clotting process. The resistance to movement of the probe 203 is sensed by the electronic drive and detection circuitry 201 and converted to electrical signal. This analog signal is processed by a microcomputer within the Sonoclot® Analyzer and output as the clot signal.

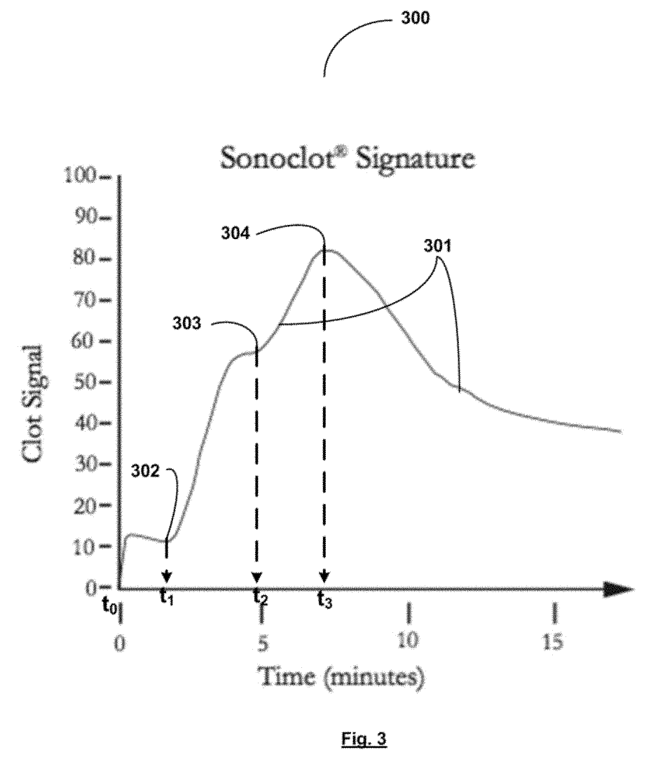

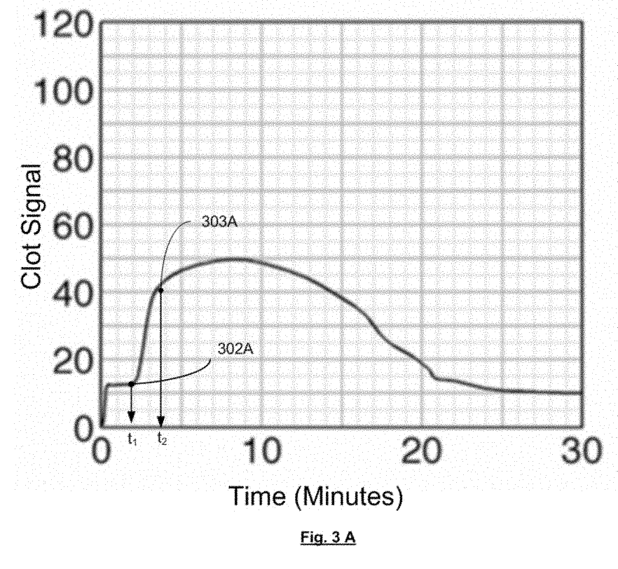

FIG. 3 is a typical trace 300 of the output signal of the Sonoclot® Analyzer using a good blood sample. As the blood sample clots, numerous mechanical changes related to the performance of the patients' hemostasis occur that alter the clot signal value. The record of the clot evolution is saved as a graph of the signal value versus time. This graph is called the Sonoclot® Signature 301. The Sonoclot® signature 301 forms the base line for any analysis or other measurements or tests by the Sonoclot® Analyzer 200.

The trace as displayed comprises the following points that are distinct and identifiable in the Sonoclot® Signature 301.

The point t1 shows when the blood sample 205 inside the cuvette 206 starts to clot. The period between start time t0 and time t1 is when the blood is in liquid state. The coagulation cascade occurs from the beginning time t0 to time t1 when the blood is in a liquid state. t1 is the time when the liquid phase ends and the viscosity of the blood sample 205 increases, that is the clotting starts, this is the activated clotting time (ACT).

Once the clotting starts the Fibrinogen in the blood converts to fibrin monomers which polymerize into a fibrin gel that increases the clot resistance as shown between time t1 and time t2. The time point t2 on the Sonoclot® Signature 301 can be considered as the end of fibrin gel formation 303. The slope of the trace in this region provides the clot rate of the blood sample 205.

The trace between the point 303 at time t2 to the point 304 at time t3 is the time when red blood cells accumulate into the fibrin and tighten the clot, further increasing the resistance on the probe 203 of the Sonoclot® Analyzer. As the clot tightens further it tend to get released from the sides of the cuvette 206 making the mechanical movement of the probe easier as indicated in the negative sloped trace of the Sonoclot® Signature 301 beyond time t3.

Sonoclot® Signature 301 beyond time starts having a downward slope after time t3 which after tightening and release from the side walls start fibrinolysis, the process of dissolution of the clot which can take a much longer time.

Fbrin clots dissolve through the activation of the fbrinolytic system. The activated enzyme plasmin is formed from plasminogen and breaks fbrin strands into smaller fbrin split products. The fbrin split products do not polymerize so as this lysing progresses, the fbrin gel dissolves breaking up the clot. With normal hemostasis the process of brinolysis occurs at much slower rates than coagulation, fibrin gel formation or clot retraction. For a normal sample full lysis will occur only after many hours.

FIG. 3A is a Sonoclot® plot of blood sample with hyperfibronolysis. Hyper fibrinolysis describes a situation with markedly enhanced fibrinolytic activity. Hyper fibronolysis limits the formation of well-defined clots. In the case of hyper fibrinolysis the clotting starts early 302A at time t1′ and proceeds through the clot formation 303A at time t2′, but the clot never thickens, but instead start to lyse before reaching full clot status.

### The Procedure

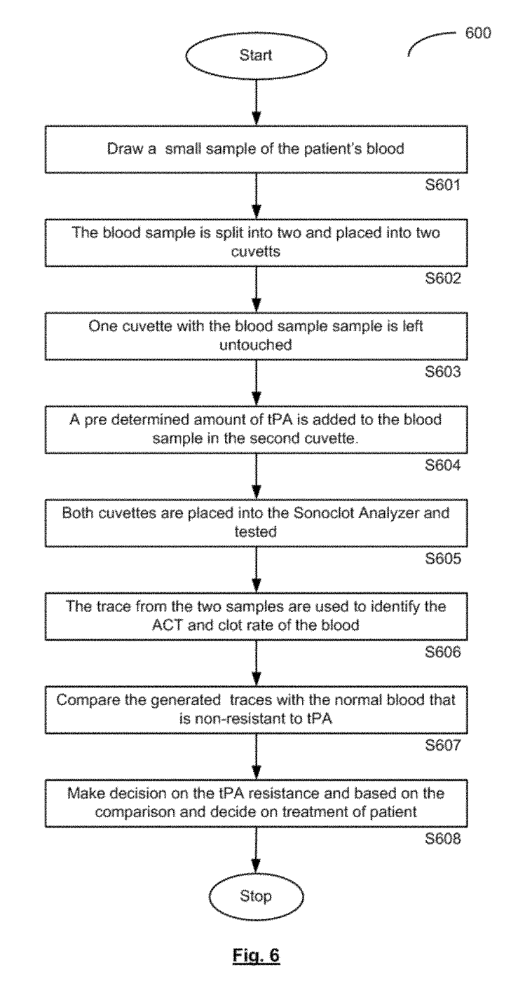

As explained above, in one embodiment, the check for the tPA resistance uses the Sonoclot® Analyzer; however, other similar machines may be used. A small amount of blood is drawn from a patient who is experiencing a stroke, heart attack, or any other problem of blood vessel clots and needs a thrombolytic to break up the clots within their blood. The process 600 of identifying if a patient's blood is resistant to tPA is shown in FIG. 6.

The process begins by drawing a very small amount of fresh blood sample, sufficient to fill two cuvettes, from the patient. S601.

The process continues by splitting the drawn blood sample into two and filling the two cuvettes. S602.

A first cuvette with the blood sample is left untouched, to provide a base line trace of the patient's blood clotting characteristics. S603. The preparation of the tPA for mixing with blood for testing described in further below.

A small pre-defined amount of the tissue plasminogen activator (tPA) is mixed into the blood sample in the second cuvette. S604.

Both the cuvettes are placed into the Sonoclot® Analyzer (or similar machine) and the clotting characteristics are tested. S605. This results in a set of traces of the clotting function of the blood as the blood in both the cuvettes clot.

The trace from the two samples are used to identify the ACT and the clot rate of the blood samples. S606.

The two traces are compared with normal blood that is non-resistant to tPA. S607. In one embodiment, the values of ACT and clot rate are extracted therefrom and those are compared with the Sonoclot® signature trace and the Sonoclot® trace of normal tPA responsive blood with the pre-determined amount of tPA added.

Based on the differences of these traces, a decision is made by the physician, on the tPA response of the patient and the start the tPA based treatment or alternate treatment for the patient. S608.

### Preparation of tPA for Efficacy Testing

Cathflo Activase (tPA) was reconstituted per instructions in the package insert to create a 2 mg of tPA/2 ml solution. This was diluted by 0.25 by adding 6 ml of normal saline to this solution.

Cathflo Activase (tPA) was then further diluted to create an equivalent physiological concentration to that seen vivo (in human blood) during standard tPA infusion dosing. This was done by taking 3.3 μL of the 0.25 solution of the standard Cathflo Activase (tPA) and adding it to 326.7 μL of freshly drawn whole blood. For the purposes of the remainder of this document we will call this solution “physiologic concentration of tPA in vivo” based on standard dosing.

### Examples

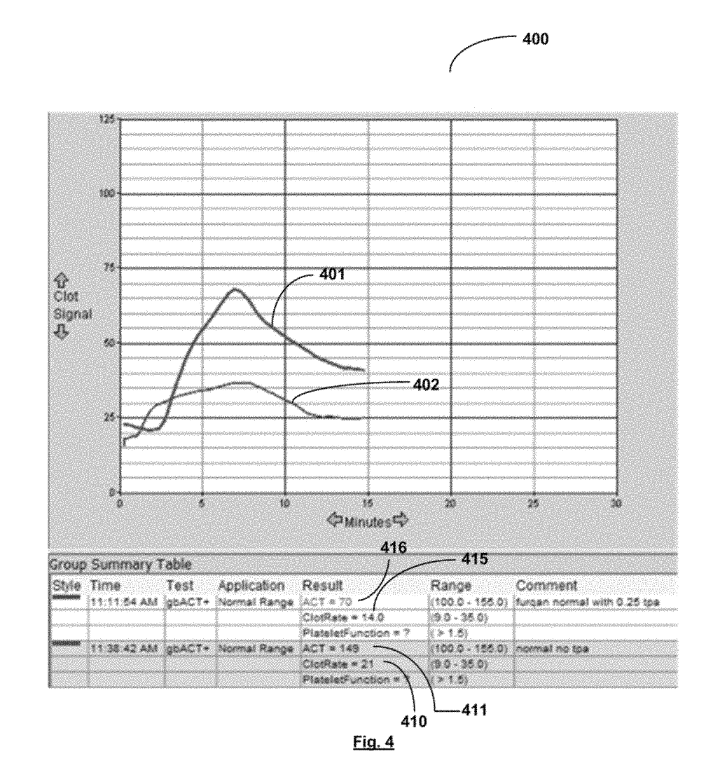

FIG. 4 shows the traces from Sonoclot® Analyzer, comparing the trace of Sonoclot® Signature 401 of normal blood of a patient without tPA resistance with the trace 402 where 3.3 μL of 0.25 dilution of standard Cathflo Activase tPA (Genentech) is added to each 326.7 μL of fresh whole blood (physiological concentration of tPA in vivo). As shown in the traces for the Sonoclot® signature 401 the ACT 410 has a value 149 with a clot rate 411 value of 21, while for the sample with tPA added the ACT 415 the value is reduced to 70 and the clot rate has dropped to 14 indicating effect of the tPA addition.

Typically a comparison of the Sonoclot® signature traces with and without tPA in concentrations that fall into the “physiologic concentration of tPA in vivo” will provide the indication of efficacy of tPA for the patient. The Sonoclot® trace with the tPA added will show a blunted curve during the clotting period (curve goes up slower and not as high). This can be used as a first indication of sensitivity of tPA.

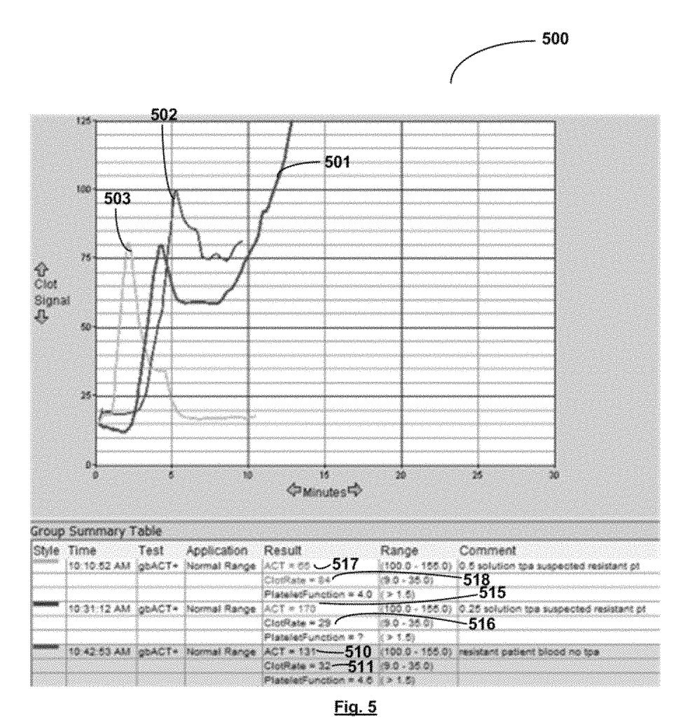

FIG. 5 is the Sonoclot® Analyzer 200 traces 500 for blood from a tPA resistant patient. The blood trace 501 without any tPA addition itself is different from the normal Sonclot signature 200 in that it has a much higher clot rate 511 having a value of 32 though the ACT 510 value is 131. The trace 502 with the addition of 0.25 dilution of standard Cathflo Activase tPA shows an ACT value 115 of 170 and a clot rate 516 of 29 which is not much lower than the initial values of ACT 510 of 131 and the clot rate 511 of 32. This is unlike the response to tPA addition response trace seen in the non-resistant tPA blood of Sonoclot® trace 402.

Sonoclot® Analyzer trace 503 is with an addition of 3.3 μL of 0.5 strength dilution of Cathflo Activase tPA to the 326.7 μL of tPA resistant blood. It is clear from the trace 503 which has ACT value of 65 and clot rate of 84 does not show a response similar to that with normal blood as in trace 402. The resistant patient shows resistance of 0.25× t-PA solution [trace 502] seen as the clot continuing to form and does not dissolve even in the presence of t-PA. A larger dose (0.5×) trace 503, is required to elicit the normal dissolution response in the resistant individual. Hence it is clear that by increasing the dose beyond what is typical, it is possible to achieve a condition of clot dissolution even in patient's blood is resistant to normal tPA concentrations. But as of now the viability of the higher dosage has not been confirmed or approved for human usage. This curve shows 0.5 strength dilution of Cathflo Activase tPA is too much of a dose to reveal resistance to tPA.

Another feature to be considered in the comparison of Sonoclot® plots of blood samples with physiologic concentration of tPA in vivo added vs the Sonoclot® plot of hyperfibrinolysis blood sample. The fibrinolysis system is responsible for removing blood clots. Hyperfibrinolysis describes a situation with markedly enhanced fibrinolytic activity. Similar to the hyperfibrinolysis the addition of tPA to the normal blood sample if it is not resistive to the drug shows indication that the tPA limits the formation of clots.

As discussed above, with the addition of tPA, the Sonoclot® plot of tPA sensitive blood tend to show some of the characteristics of the hyperfibrinolysis. Looking at the Sonoclot® plot 402 in FIG. 4 it is clear that with the tPA addition, even though the clotting starts earlier, the clot never matures but starts to lyse early providing a more blunted curve similar to that expected of hyperfibrinolysis. Hence by comparison of the clotting characteristics of the blood sample (start time, rate, peak value, etc.) with and without tPA addition and the comparison of the shapes of the two Sonoclot® plots, is used to arrive at a definitive decision on the efficacy of the treatment of the patient using tPA.

A study and comparison of the Sonoclot® Analyzer traces 400 and 500 are done to find the comparative differences. The identified differences in the characteristic values and the shape of the plots provide a clear indication as to whether the patient is resistant to tPA to decide if the patient should be treated with tPA or be directed for alternate treatment.

Therefore, a method that predicts tPA resistance prior to administration has been developed to help the doctors decide on an appropriate and optimized treatment for patients experiencing a stroke, heart attack, or any blood vessel clots.

While the invention has been described in terms of an embodiments, those of ordinary skill in the art will recognize that the invention is not limited to the embodiment described, but can be practiced with modification and alteration within the spirit and scope of the appended claims. The description is thus to be regarded as illustrative instead of limiting. There are numerous other variations to different aspects of the invention described above, which in the interest of conciseness have not been provided in detail. Accordingly, other embodiments are within the scope of the claims.

The invention has been described in relation to particular example, relating to treatment of thrombotic problems by tPA, which is intended in all respects to be illustrative rather than restrictive. Those skilled in the art will appreciate that many different drugs, used for thrombotic disorders, that will benefit from modification of the present invention.

As will be understood by those familiar with the art, the invention may be embodied in other specific forms without departing from the spirit or essential characteristics thereof. Likewise, the particular naming and division of the members, features, attributes, and other aspects are not mandatory or significant, and the mechanisms that implement the invention or its features may have different structural construct, names, and divisions. Accordingly, the disclosure of the invention is intended to be illustrative, but not limiting, of the scope of the invention.

While the invention has been described in terms of several embodiments, those of ordinary skill in the art will recognize that the invention is not limited to the embodiments described, but can be practiced with modification and alteration within the spirit and scope of the appended claims. The description is thus to be regarded as illustrative instead of limiting. There are numerous other variations to different aspects of the invention described above, which in the interest of conciseness have not been provided in detail. Accordingly, other embodiments are within the scope of the claims.

The invention has been described in relation to particular examples, which are intended in all respects to be illustrative rather than restrictive. Those skilled in the art will appreciate that many different combinations will be suitable for practicing the present invention. Other implementations of the invention will be apparent to those skilled in the art from consideration of the specification and practice of the invention disclosed herein. Various aspects and/or components of the described embodiments may be used singly or in any combination. It is intended that the specification and examples be considered as exemplary only, with a true scope and spirit of the invention being indicated by the following claims.

What is claimed is:

1. A method comprising:
drawing an amount of a fresh blood sample from a patient sufficient to fill two cuvettes;
splitting the drawn blood sample into two blood sample portions and filling the two cuvettes with the two blood sample portions;
mixing a small pre-defined amount of the tissue plasminogen activator (tPA) into one of the two cuvettes;
placing the two cuvettes into a blood coagulation tester to test clotting characteristics of the two sample blood portions, wherein a set of traces of the clotting function of blood as the blood sample portions in the two cuvettes clot is generated;
comparing the set of traces with normal blood that is non-resistant to tPA; and
determining a tPA response of the patient based on the comparing.

2. The method of claim 1, wherein the blood coagulation tester comprises a Sonoclot® Analyzer.

3. The method of claim 1, wherein the set of traces are used to identify the activated clotting time (ACT) and the clot rate of the blood sample portions.

4. The method of claim 1, wherein values of ACT and clot rate are extracted from the set of traces.

5. The method of claim 1, wherein the set of traces are compared with a Sonoclot® signature trace.

6. The method of claim 1, wherein the set of traces are compared with a Sonoclot® signature trace and a Sonoclot® trace of normal tPA responsive blood with a pre-determined amount of tPA added.

7. The method of claim 1, further comprising preparing the tPA for efficacy testing.

8. The method of claim 7, wherein the preparing the tPA for efficacy testing comprises:
reconstituting cathflo activase; and
diluting the solution by adding normal saline.

9. The method of claim 8, wherein the reconstituting cathflo activase as per manufacturers' instructions to create a 2 mg of tPA/2 mL solution.

10. The method of claim 8, wherein the diluting the solution dilutes the solution by 0.25, and wherein adding normal saline comprises adding 6 mL of normal saline to 2 ml of reconstituted solution.

11. The method of claim 10, wherein mixing a small pre-defined amount of the tissue plasminogen activator (tPA) into one of the two cuvettes comprises adding 3.3 μL of the 0.25 diluted solution to 326.7 μL of the one of the two blood sample portions in the one of the two cuvettes.

12. The method of claim 1, wherein the fresh blood sample is drawn from the patient when they are experiencing a blood vessel clot.

13. The method of claim 12, wherein the blood vessel clot comprises a heart attack.

14. The method of claim 12, wherein the blood vessel clot comprises a stroke.

15. The method of claim 12, wherein the blood vessel clot comprises one selected from the group consisting of a pulmonary embolism (PE), a myocardial infarction (MI), an arterial thrombosis, and a deep vein thrombosis (DVT).

16. The method of claim 12, wherein the patient is experiencing one selected from the group of a Pulmonary Embolism (PE), a myocardial infarction (MI), an arterial thrombosis, and a deep vein thrombosis (DVT) related to the blood vessel clot.

17. The method of claim 1, further comprising administering an effective amount of tPA if the tPA response is that the patient is not thrombolytic resistant.

18. The method of claim 1, further comprising performing an alternate interventional procedure if the tPA response is that the patient is thrombolytic resistant.

19. A blood coagulation tester comprising:
a first cuvette for receiving a normal blood sample from a patient;
a second cuvette for receiving a mixture of tPA and a blood sample the patient;
a probe in each of the first cuvette and the second cuvette;
an electomechanical transducer coupled to each of the probes in the first cuvette and the second cuvette to move the probes in the first and second cuvettes;
electronic drive and detection circuitry coupled to each of the electromechanical transducers configured to cause each of the electromechanical transducers to move the probes in the first and second cuvettes and to measure resistance to movement of the probes in the first and second cuvettes; and
a microcomputer coupled to the electronic drive and detection circuitry to process the measured resistance and output a set of traces indicating the clotting function of the normal blood sample in the first cuvette and the mixture of the tPA and the blood sample in the second cuvette, wherein the set of traces are configured to be compared to determine whether the blood sample is thrombolytic resistant.

20. The blood coagulation tester of claim 19, wherein the blood coagulation tester comprises a Sonoclot® Analyzer.

21. The blood coagulation tester of claim 19, wherein the blood coagulation tester comprises a Thrombo Elastography (TEG®) or a Rotational thromboelastrometry (ROTEM®).
